(19) United States
(12) Patent Application Publication
(10) Pub. No.: US 2004/0236515 A1
(43) Pub. Date: Nov. 25, 2004

(54) System, method and computer product for predicting protein- protein interactions

(75) Inventors: Ming Zhao, Clifton Park, NY (US); Joshua Michael Temkin, Clifton Park, NY (US)

Correspondence Address: General Electric Company, GRC Patent Docket Rm 4A59, P.O. Box 8, Bldg. K-1, Schenectady, NY, 12301, US

(73) Assignee: General Electric Company

(21) Appl. No.: 10/442,730

(22) Filed: May 20, 2003

(51) Int. Cl. G06F 19/00; G01N 33/48; G01N 33/50

(52) U.S. Cl. 702/19

(57) ABSTRACT

System, method and computer product for predicting protein-protein interactions. In this disclosure, there is at least one biological data source containing biological data. A protein-protein interactions prediction module accesses the at least one biological data source and predicts interactions with a target protein having known domain and motif information. The prediction module includes a search module that searches the at least one biological data source for a set of proteins with domain and motif information similar to the domain and motif information of the target protein. An extraction module extracts interaction proteins from the set of proteins. A cellular location module determines whether the extracted interaction proteins are co-located at the same cellular location as the target protein. An analysis module determines protein-protein interactions of the interaction proteins with the target protein based on interaction domains and motifs and cellular location.

### BACKGROUND OF THE INVENTION

This disclosure relates generally to bioinformatics and more particularly to predicting protein-protein interactions.

Proteins have a significant role in regulating cellular events such as signal transduction, cell cycle, protein trafficking, targeted proteolysis, cytoskeletal organization and gene regulation/expression/translation. Pathways that make these events happen are linked by protein-protein interactions. Generally, protein-protein interactions are direct and specific molecular interactions among proteins that control biological processes. Researchers are interested in understanding protein-protein interactions because the interactions can lead to novel therapeutic and diagnostic development opportunities.

Determining protein-protein interactions has typically been a slow and cumbersome process. For example, the most commonly used approach involves yeast two-hybrid systems, which rely on a reporter gene (e.g. gall-lacZ—the beta-galactosidase gene) that is transcriptionally activated to establish relations between two or more proteins of a given genome. There will be a color reaction on specific media if there is a protein-protein interaction. The yeast two-hybrid systems generally work well, but require careful experimental set-up and can result in false positives (i.e., indications of protein-protein interactions when in reality there are none). In addition, the yeast two-hybrid systems can take several years to produce full protein-protein interaction maps.

Until recently it was only possible to determine protein-protein interactions by applying “wet lab” techniques like the yeast two-hybrid systems. There are now several computational techniques that have emerged that enable researchers to predict protein-protein interactions. One of these computational techniques involves using amino acid sequences to predict protein-protein interactions. In particular, if two sets of proteins share conserved sequences, then the protein in one set may interact with the ones in the other protein set. A problem with this approach is that this technique is limited to proteins that have known structures and will not work well with proteins where prior protein knowledge is not available. Some newer computational techniques have addressed the problem of predicting interactions for proteins that do not have prior protein knowledge available, however, these approaches are subject to a high rate of false positives.

Therefore, there is a need for a computational approach that can predict protein-protein interactions with a reduced amount of false positives in order to facilitate novel therapeutic and diagnostic development opportunities.

### BRIEF DESCRIPTION OF THE INVENTION

In a first embodiment of this disclosure, there is a method and computer readable medium that stores instructions for instructing a computer system to predict protein-protein interactions. This embodiment comprises receiving a target protein having known domain and motif information; finding a set of proteins with domain and motif information similar to the domain and motif information of the target protein; extracting interaction proteins from the set of proteins; determining whether the interaction proteins are co-located at the same cellular location as the target protein; and predicting protein-protein interactions with the target protein based on interaction domains and motifs and cellular location.

In another embodiment of this disclosure, there is a method and computer readable medium that stores instructions for instructing a computer system to predict protein-protein interactions. This embodiment comprises receiving a target protein having known domain and motif information; in response to the received target protein, searching at least one biological data source; finding a set of proteins with domain and motif information similar to the domain and motif information of the target protein; extracting interaction proteins from the set of proteins; determining whether the interaction proteins are co-located at the same cellular location as the target protein; predicting protein-protein interactions of the interaction proteins with the target protein based on interaction domains and motifs and cellular location; and providing an indication of a protein-protein interaction if an interaction protein has interaction domains and motifs with the target protein and similar cellular location.

In a third embodiment of this disclosure, there is a system for predicting protein-protein interactions. In this system there is at least one biological data source containing biological data. A protein-protein interaction prediction module is configured to access the at least one biological data source and predict interactions with a target protein having known domain and motif information. The prediction module comprises a search module that searches the at least one biological data source for a set of proteins with domain and motif information similar to the domain and motif information of the target protein. An extraction module extracts interaction proteins from the set of proteins. A cellular location module determines whether the extracted interaction proteins are co-located at the same cellular location as the target protein. An analysis module determines protein-protein interactions of the interaction proteins with the target protein based on interaction domains and motifs and cellular location.

### BRIEF DESCRIPTION OF THE DRAWINGS

FIG. 1 shows a schematic of a general-purpose computer system in which a system that predicts protein-protein interactions operates;

FIG. 2 shows a high level diagram of a system that predicts protein-protein interactions, which operates on the computer system shown in FIG. 1;

FIG. 3 shows a flow chart describing the operations performed by the system shown in FIG. 2;

FIG. 4 shows an illustrative example of predicting a protein-protein interaction with the system shown in FIG. 2;

FIG. 5 shows an architectural diagram of a system for implementing the protein-protein interactions prediction module shown in FIG. 2 on a network; and

FIG. 6 shows an implementation of the system shown in FIG. 2 within a pathway generation system.

### DETAILED DESCRIPTION OF THE INVENTION

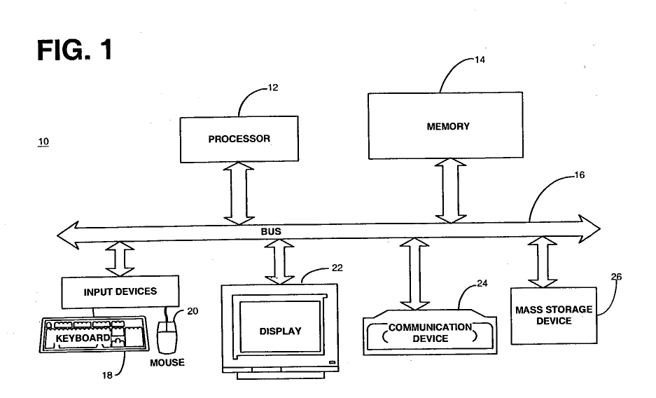

FIG. 1 shows a schematic of a general-purpose computer system 10 in which a system that predicts protein-protein interactions operates. The computer system 10 generally comprises at least one processor 12, memory 14, input/output devices, and a bus 16 connecting the processor, memory and input/output devices. The processor 12 accepts instructions and data from the memory 14 and performs various data processing functions like searching data sources, data extraction and data analysis. The processor 12 includes an arithmetic logic unit (ALU) that performs arithmetic and logical operations and a control unit that extracts instructions from memory 14 and decodes and executes them, calling on the ALU when necessary. The memory 14 generally includes a random-access memory (RAM) and a read-only memory (ROM), however, there may be other types of memory such as programmable read-only memory (PROM), erasable programmable read-only memory (EPROM) and electrically erasable programmable read-only memory (EEPROM). Also, the memory 14 preferably contains an operating system, which executes on the processor 12. The operating system performs basic tasks that include recognizing input, sending output to output devices, keeping track of files and directories and controlling various peripheral devices.

The input/output devices may comprise a keyboard 18 and a mouse 20 that enter data and instructions into the computer system 10. Also, a display 22 may be used to allow a user to see what the computer has accomplished. Other output devices may include a printer, plotter, synthesizer, speakers, and other devices. A communication device 24 such as a telephone or cable modem or a network card such as an Ethernet adapter, local area network (LAN) adapter, integrated services digital network (ISDN) adapter, or Digital Subscriber Line (DSL) adapter, enables the computer system 10 to access other computers and resources on a network such as a LAN, a wide area network (WAN) or the Internet. A mass storage device 26 may be used to allow the computer system 10 to permanently retain large amounts of data. The mass storage device may include all types of disk drives such as floppy disks, hard disks and optical disks, as well as tape drives that can read and write data onto a tape that could include digital audio tapes (DAT), digital linear tapes (DLT), or other magnetically coded media.

The above-described computer system 10 can take the form of a hand-held digital computer, personal digital assistant computer, notebook computer, personal computer, workstation, mini-computer, mainframe computer or supercomputer.

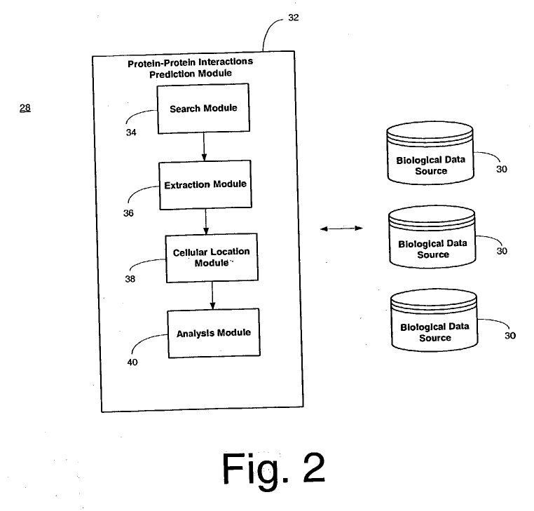

FIG. 2 shows a high level diagram of a system 28 that predicts protein-protein interactions, which operates on the computer system 10 shown in FIG. 1. The protein-protein interactions prediction system 28 comprises at least one biological data source 30 containing biological data which may include data relating to gathering, analyzing, and representing proteins, along with their structure and function. An illustrative, but non-exhaustive list of biological data sources may include protein databases such as BLAST (protein sequence and structural information), Pfam (collection of multiple sequence alignments covering many protein domains/motifs and families), SwissProt (annotated protein sequences) GenBank, RefSeq, and LocusLink.

The system 28 also comprises a protein-protein interactions prediction module 32 configured to access the biological data sources 30 and predict interactions with a target protein having known domain and motif information. Domain information comprises a region of special biological interest within a single protein sequence. One of ordinary skill in the art will recognize a domain may also be defined as a compact, locally folded region within the three-dimensional structure of a protein that may encompass regions of several distinct protein sequences that accomplishes a specific function. Motif information comprises a conserved element of a protein sequence alignment that usually correlates with a particular function. Motifs are typically generated from a local multiple protein sequence alignment corresponding to a region whose function or structure is known. In more specific words, a motif is a small number of amino acids that form a particular pattern with biological function.

The protein-protein interactions prediction module 32 comprises a search module 34 that searches the biological data sources 30 for proteins with domain and motif information similar to the domain and motif information of the target protein. Each of the above-mentioned biological data sources contains information about proteins and the domains that comprise them. In addition, these biological data sources contain information about what proteins interact with each other, and of the proteins that interact, what domains are binding (sticking) to each other. These interacting domains are also stored in the data sources. In order to search for proteins with similar domains and motifs, the search module 34 queries the biological data sources 30 using an identifier (e.g., an accession number) of the target protein. The search module 34 then returns a set of domains believed to be contained within the queried protein. The search module 34 then automatically generates another query that uses this set of domains as the query terms of the data sources 30 to look for other proteins with similar domain profiles. This returns a set of proteins that have similar domain and motif characteristics as the target protein.

An extraction module 36 extracts interaction proteins from the proteins found to have domain and motif information similar to the domain and motif information of the target protein. Interaction proteins are proteins that have domain and motif information that interact with the proteins found to have domain and motif information that is similar to the domain and motif information of the target protein. The domain and motif information from the interaction proteins that interact with the domain and motif information of the proteins are referred to as interaction domains and interaction motifs, respectively. The extraction module 36 extracts interaction proteins by using the set of proteins returned from the similarity search performed by the search module 34. In particular, the extraction module 36 uses this set of proteins as search terms to extract interaction proteins from an interaction database stored in the biological data sources 30. More specifically, the extraction module 36 queries individually each protein returned in the set from the similarity search against the biological data sources 30 for other proteins for which interactions have been found and stored in the data sources. The extraction module 36 then returns a set of corresponding interacting proteins as results from this query.

A cellular location module 38 determines whether the extracted interaction proteins are co-located at the same cellular location as the target protein. The cellular location module 38 is able to determine whether the extracted interaction proteins are co-located at the same cellular location as the target protein by referring to the proteins' attributes. Each protein has a set of attributes assigned to it and cellular location is one such attribute. The biological data sources 30 typically stores these attributes in its relational schema. The cellular location module 38 finds these attributes by using queries. The cellular location module 38 uses one query to return the attribute data from the cellular location tables as a list of locations known to contain the protein of interest. The cellular location module 38 then uses another query to determine co-location by comparing the attributes of two or more proteins together. If both or the set of proteins being queried contain the same attributes, in this case cellular location, the cellular location module 38 indicates that they are co-located.

An analysis module 40 determines protein-protein interactions of the interaction proteins with the target protein based on the interaction domain and motif information and cellular location. The analysis module 40 determines protein-protein interactions by comparing the domains of the target protein with the domains from the set of proteins returned from the searches performed by the search module 34, extraction module 36 and cellular location module 38 against the database of known domains and motifs and domain and motif interactions. The analysis module 40 will predict that the target protein will interact with one of the other proteins if they are found to have at least one pair of domains that interact based on the domains and motif and domain and motif interaction table in the biological data sources. For example, if target protein X has domains A and B and potential interacting protein Y has domains C and D, and if in the domain and motif and domain and motif interaction table it is known previously (such as a paper that describes these interactions) that domain A binds to domain C and given the fact that target X and potential interaction protein Y are co-located, then the analysis module 40 predicts that X and Y interact.

The analysis module 40 also generates a confidence value for each determined protein-protein interaction. A confidence value is a value indicative of the belief that a protein-protein interaction exists. The analysis module 40 determines confidence values by using a weighting distribution of attributes that are met in predicting the interaction. For example, predictions in which all of the attributes such as collocation, coexpression, tissue distribution have highly correlated domain pair interactions will be scored higher than other predictions in which only a few of these conditions are met. Those that meet none of the described conditions are deemed of low value and are scored as such.

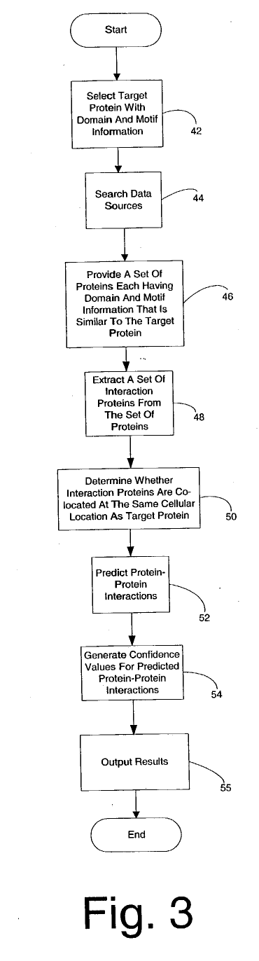

FIG. 3 shows a flow chart describing the operations performed by the system shown in FIG. 2. At 42, the user of the system 28 selects a target protein having known domain and motif information. The search module then initiates a search of the biological data sources at 44. In particular, the search module searches the data sources for proteins having domain and motif information that is similar to the domain and motif information of the target protein. The search module then provides a candidate set of proteins that each has domain and motif information similar to the domain and motif information of the target protein at 46. The extraction module then extracts a set of interaction proteins from the candidate set at 48. As mentioned above, the interaction proteins each have domain and motif information that interact with the candidate set of proteins. The cellular location module then determines whether each of the interaction proteins in the candidate set is co-located at the same cellular location as the target protein at 50. In particular, the cellular location module determines the specific cellular location at which the interaction protein and its accompanying domain and motif information are obtained from. For example, one interaction protein can be taken from a lung tissue, while another one could be from a brain tissue. The analysis module then predicts protein-protein interactions with the target protein based on interaction domain and motif information and cellular location at 52. For example, if an interaction protein has interaction domain and motif information with the target protein and both are co-located at the same cellular location (e.g., large intestine), then the analysis module would designate a protein-protein interaction. On the other hand, if the interaction protein and target protein had interaction domain and motif information but one was taken from a pancreas tissue while the other is from a lung tissue, then the analysis module would not indicate that an interaction exists. In addition, the analysis module generates a confidence value for all of the designated protein-protein interactions at 54. The system 28 then presents the results to the user at 55.

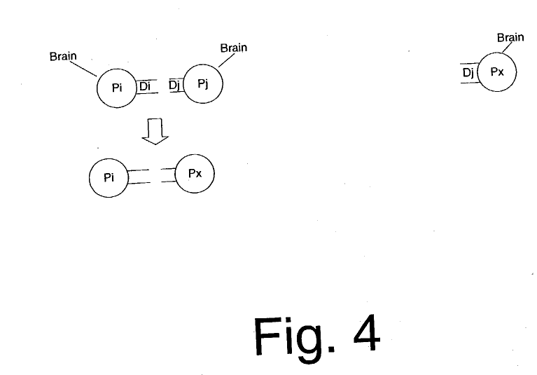

FIG. 4 shows an illustrative example of predicting a protein-protein interaction with the system shown in FIG. 2. This example is for illustrative purposes and one of ordinary skill will recognize that there are other ways to show domain and motif information of a protein. For ease of explanation, the proteins shown in FIG. 4 contain only domain information, but usually would have more information such as the motif. FIG. 4 shows proteins Pi and Pj taken from a set of proteins P having proteins p1, p2, . . . ,Pi, . . . Pj, . . . Pn and a target protein Px. Proteins Pi, Pj and Px are taken from a tissue, for example, brain. FIG. 4 also shows that proteins Pi and Pj have domain information Di and Dj, respectively, wherein Di and Dj are interaction domains. Target protein Px is known to have domain information Dj which is similar to the domain information of protein Pj. Since Px and Pj have similar domain information (Dj) and Px and Pi are co-located at the same cellular location (brain), then the protein-protein interactions prediction module 32 would predict that Pi interacts with Px. Note the result would be different if the target protein Px and Pi were located at different cellular locations (e.g., brain and stomach). That is, the protein-protein interactions prediction module 32 would not predict an interaction because of the different cellular locations of Px and Pi.

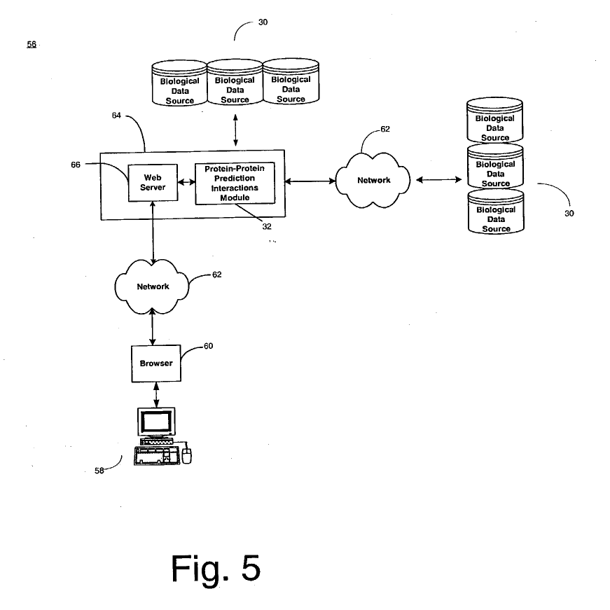

FIG. 5 shows an architectural diagram of a system 56 for implementing the protein-protein interactions prediction module shown in FIG. 2 on a network. In FIG. 5, a computing unit 58 allows a user to access the protein-protein interactions prediction module 32 and the biological data sources 30 over a network such as the Internet. The computing unit 58 can take the form of a hand-held digital computer, personal digital assistant computer, notebook computer, personal computer or workstation. The user uses a web browser 60 such as Microsoft INTERNET EXPLORER™ Netscape NAVIGATOR™ or Mosaic to locate, display and use the protein-protein interactions prediction module 32 and the biological data sources 30 on the computing unit 58. A communication network 62 such as an electronic or wireless network connects the computing unit 58 to the protein-protein interactions prediction module 32 including the biological data sources 30. In particular, the computing unit 58 may connect to the protein-protein interactions prediction module 32 and the data sources 30 through a private network such as an extranet or intranet or a global network such as a WAN (e.g., the Internet) or through share drives, databases and subscription services. As shown in FIG. 5, the protein-protein interactions prediction module 32 may reside in a server 64, which comprises a web server 66 that serves the protein-protein interactions prediction module 32 and the biological data sources 30. One of ordinary skill in the art will recognize that the protein-protein interactions prediction module 32 does not have to be co-resident with the server 64. Also, the protein-protein interactions prediction module 32 may also connect to the biological data sources over another network. In addition, the protein-protein interactions prediction module 32 may be distributed over more than one server or other configurations of networked devices.

If desired, the system 56 may have functionality that enables authentication and access control of users accessing the protein-protein interactions prediction module 32 and data sources 30. Both authentication and access control can be handled at the web server level by the protein-protein interactions prediction module 32 itself, or by commercially available packages such as Netegrity SITEMINDER. Information to enable authentication and access control such as the user's name, location, telephone number, organization, login identification, password, access privileges to certain resources, physical devices in the network, services available to physical devices, etc. can be retained in a database directory. The database directory can take the form of a lightweight directory access protocol (LDAP) database; however, other directory type databases with other types of schema may be used including relational databases, object-oriented databases, flat files, or other data management systems.

In this implementation, the protein-protein interactions prediction module 32 may run on the web server 66 in the form of servlets, which are applets (e.g., Java applets) that run a server. Alternatively, the protein-protein interactions prediction module 32 may run on the web server 66 in the form of CGI (Common Gateway Interface) programs. The servlets access the biological data sources 30 using JDBC or Java database connectivity, which is a Java application programming interface that enables Java programs to execute SQL (structured query language) statements. Alternatively, the servlets may access the data sources 30 using ODBC or open database connectivity. Using hypertext transfer protocol or HTTP, the web browser 60 obtains a variety of applets that execute the protein-protein interactions prediction module 32 on the computing unit 58 allowing the user to perform processing operations discussed below. Also, the web browser may be used to view Web pages containing biological data and access analysis tools, plotting tools, graphics programs, etc.

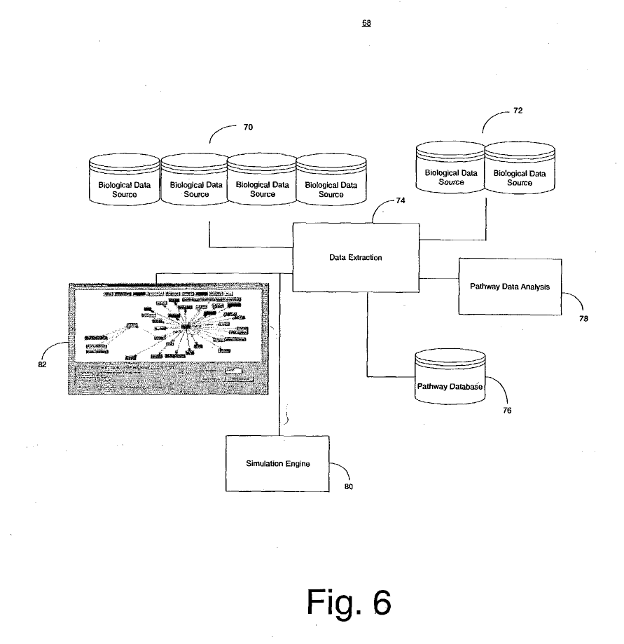

FIG. 6 shows an implementation of the protein-protein interactions prediction system 28 shown in FIG. 2 within a pathway generation system 68. The pathway generation system 68 is in communication with biological data sources 70 and 72. Biological data sources 70 contain data such as protein interaction data and biological data sources 72 contain data such as textual information on proteins and protein sequences. An illustrative but non-exhaustive list of examples of protein interactive data sources not including the aforementioned sources are Pronet, BIND and Transpath and a non-exhaustive list of examples of data sources containing textual information on proteins are Swiss Prot and PubMed. A data extraction module 74 automatically extracts biological data from the data sources 70 and 72. A pathway database 76 stores the biological data retrieved by the data extraction module 74. In addition to the protein interactions, annotated protein sequences and textual information retrieved from the Pronet, BIND, Transpath, Swiss Prot, and PubMed databases, the pathway database 76 may store other types of data. The pathway generation system 68 also comprises a pathway data analysis module 78 that assimilates the biological data stored in the pathway database 76 into a hypotheses prediction for generating a pathway. The pathway data analysis module 78 includes the protein-protein interactions prediction module of this disclosure. Also included in the pathway generation system 68 is a simulation engine 80 that enables, among other things, generation of pathways based upon prediction and other data. A visualization module 82 generates a visual representation of the pathway generated by the pathway data analysis module 78. U.S. patent application Ser. No. 10/307,556 entitled System, Method and Computer Product For Predicting Biological Pathways, filed on Dec. 2, 2002, provides a more detailed description of the pathway generation system 68.

The foregoing figures show one embodiment of the functionality and operation of the protein-protein interactions prediction system. In this regard, some of the blocks represent a module, component, segment, or portion of code, which comprises one or more executable instructions for implementing the specified logical function(s). It should also be noted that in some alternative implementations, the functions noted in the blocks may occur out of the order noted in the figure or, for example, may in fact be executed substantially concurrently or in the reverse order, depending upon the functionality involved. Furthermore, the functions can be implemented in programming languages such as Java, however, other languages can also be used.

The above-described systems comprise an ordered listing of executable instructions for implementing logical functions. The ordered listing can be embodied in any computer-readable medium for use by or in connection with a computer-based system that can retrieve the instructions and execute them. In the context of this application, the computer-readable medium can be any means that can contain, store, communicate, propagate, transmit or transport the instructions. The computer readable medium can be an electronic, a magnetic, an optical, an electromagnetic, or an infrared system, apparatus, or device. An illustrative, but non-exhaustive list of computer-readable mediums can include an electrical connection (electronic) having one or more wires, a portable computer diskette (magnetic), a random access memory (RAM) (magnetic), a read-only memory (ROM) (magnetic), an erasable programmable read-only memory (EPROM or Flash memory) (magnetic), an optical fiber (optical), and a portable compact disc read-only memory (CDROM) (optical).

The computer readable medium may comprise paper or another suitable medium upon which the instructions are printed. For instance, the instructions can be electronically captured via optical scanning of the paper or other medium, then compiled, interpreted or otherwise processed in a suitable manner if necessary, and then stored in a computer memory.

It is apparent that there has been provided a system, method and computer product for predicting protein-protein interactions. While the invention has been particularly shown and described in conjunction with a preferred embodiment thereof, it will be appreciated that variations and modifications can be effected by a person of ordinary skill in the art without departing from the scope of the invention.

What is claimed is:

1. A method for predicting protein-protein interactions, comprising:
receiving a target protein having known domain and motif information;
finding a set of proteins with domain and motif information similar to the domain and motif information of the target protein;
extracting interaction proteins from the set of proteins;
determining whether the interaction proteins are co-located at the same cellular location as the target protein; and
predicting protein-protein interactions with the target protein based on interaction domains and motifs and cellular location.

2. The method according to claim 1, further comprising generating a confidence value with each predicted protein-protein interaction, wherein the confidence value is indicative of the belief that a protein-protein interaction exists.

3. The method according to claim 1, wherein the finding of a set of proteins with domain and motif information similar to the domain and motif information of the target protein comprises searching a plurality of biological data sources each containing protein information.

4. The method according to claim 1, wherein the finding of a set of proteins with domain and motif information similar to the domain and motif information of the target protein comprises searching a pathway database containing extracted biological pathway informatics.

5. The method according to claim 1, wherein the predicting of protein-protein interactions comprises generating an indication of a protein-protein interaction if an interaction protein has interaction domains and motifs with the target protein and are co-located at the same cellular location.

6. A method for predicting protein-protein interactions, comprising:
inputting a target protein having known domain and motif information;
searching at least one biological data source;
finding a set of proteins with domain and motif information similar to the domain and motif information of the target protein;
extracting interaction proteins from the set of proteins;
determining whether the interaction proteins are co-located at the same cellular location as the target protein; and
predicting protein-protein interactions with the target protein based on interaction domains and motifs and cellular location.

7. The method according to claim 6, further comprising generating a confidence value with each predicted protein-protein interaction, wherein the confidence value is indicative of the belief that a protein-protein interaction exists.

8. The method according to claim 6, wherein the predicting of protein-protein interactions comprises generating an indication of a protein-protein interaction if an interaction protein has interaction domains and motifs with the target protein and are co-located at the same cellular location.

9. A method for enabling prediction of protein-protein interactions over a network, comprising:
receiving a target protein having known domain and motif information;
in response to the received target protein, searching at least one biological data source;
finding a set of proteins with domain and motif information similar to the domain and motif information of the target protein;
extracting interaction proteins from the set of proteins;
determining whether the interaction proteins are co-located at the same cellular location as the target protein;
predicting protein-protein interactions with the target protein based on interaction domains and motifs and cellular location; and
providing an indication of a protein-protein interaction if an interaction protein and the target protein have interaction domains and motifs and similar cellular location.

10. The method according to claim 9, further comprising generating a confidence value with each predicted protein-protein interaction, wherein the confidence value is indicative of the belief that a protein-protein interaction exists.

11. A system for predicting protein-protein interactions, comprising:
at least one biological data source containing biological data; and
a protein-protein interaction prediction module configured to access the at least one biological data source and predict interactions with a target protein having known domain and motif information, the prediction module comprising:
a search module that searches the at least one biological data source for a set of proteins with domain and motif information similar to the domain and motif information of the target protein;
an extraction module that extracts interaction proteins and associated interaction domains and motifs from the set of proteins;
a cellular location module that determines whether the extracted interaction proteins are co-located at the same cellular location as the target protein; and
an analysis module that determines protein-protein interactions of the interaction proteins with the target protein based on the interaction domains and motifs and cellular location.

12. The system according to claim 11, wherein the analysis module comprises a confidence module that generates a confidence value with each predicted protein-protein interaction, wherein the confidence value is indicative of the belief that a protein-protein interaction exists.

13. The system according to claim 11, wherein the at least one biological data source comprises a pathway database containing extracted biological pathway informatics.

14. The system according to claim 11, wherein the analysis module generates an indication of a protein-protein interaction if an interaction protein and the target protein have interaction domains and motifs and are co-located at the same cellular location.

15. A computer-readable medium storing computer instructions for instructing a computer system to predict protein-protein interactions, the computer instructions comprising:
receiving a target protein having known domain and motif information;
finding a set of proteins with domain and motif information similar to the domain and motif information of the target protein;
extracting interaction proteins from the set of proteins;
determining whether the interaction proteins are co-located at the same cellular location as the target protein; and
predicting protein-protein interactions with the target protein based on interaction domains and motifs and cellular location.

16. The computer-readable medium according to claim 15, further comprising instructions for generating a confidence value with each predicted protein-protein interaction, wherein the confidence value is indicative of the belief that a protein-protein interaction exists.

17. The computer-readable medium according to claim 15, wherein the finding of a set of proteins with domain and motif information similar to the domain and motif information of the target protein comprises instructions for searching a plurality of biological data sources each containing protein information.

18. The computer-readable medium according to claim 15, wherein the finding of a set of proteins with domain and motif information similar to the domain and motif information of the target protein comprises instructions for searching a pathway database containing extracted biological pathway informatics.

19. The computer-readable medium according to claim 15, wherein the predicting of protein-protein interactions comprises instructions for generating an indication of a protein-protein interaction if an interaction protein has interaction domains and motifs with the target protein and are co-located at the same cellular location.

20. A computer-readable medium storing computer instructions for instructing a computer system to enable prediction of protein-protein interactions over a network, the computer instructions comprising:
receiving a target protein having known domain and motif information;
in response to the received target protein, searching at least one biological data source;
finding a set of proteins with domain and motif information similar to the domain and motif information of the target protein;
extracting interaction proteins from the set of proteins;
determining whether the interaction proteins are co-located at the same cellular location as the target protein;
predicting protein-protein interactions of the interaction proteins with the target protein based on interaction domains and motifs and cellular location; and
providing an indication of a protein-protein interaction if an interaction protein has interaction domains and motifs with the target protein and are co-located at the same cellular location.

21. The computer-readable medium according to claim 20, further comprising instructions for generating a confidence value with each predicted protein-protein interaction, wherein the confidence value is indicative of the belief that a protein-protein interaction exists.
